(19) United States
(12) Patent Application Publication
(10) Pub. No.: US 2025/0295465 A1
(43) Pub. Date: Sep. 25, 2025

(54) UNIVERSAL POLE MOUNTING BRACKETS FOR MEDICAL DEVICES

(71) Applicant: Terumo Cardiovascular Systems Corporation, Ann Arbor, MI (US)

(72) Inventors: Daniel James Cooney, Northville, MI (US); Rogelio Insignares, Howell, MI (US)

(21) Appl. No.: 19/078,824

(22) Filed: Mar. 13, 2025

Related U.S. Application Data
(60) Provisional application No. 63/567,278, filed on Mar. 19, 2024.

(51) Int. Cl. A61B 50/10

(52) U.S. Cl. CPC A61B 50/10

(57) ABSTRACT

Bracket assemblies can be used to pole mount medical equipment such as blood parameter monitoring devices, extracorporeal blood pumps, and other medical devices. The use of such pole mount bracket assemblies can serve to minimize congestion of areas around patients receiving medical attention. In some examples, the pole mount bracket assemblies can be made to be easily adjustable and to be readily adaptable to firmly clamp to poles of various diameters.

### CROSS-REFERENCE TO RELATED APPLICATIONS

This application claims the benefit of U.S. Provisional Application Ser. No. 63/567,278 filed Mar. 19, 2024. The disclosure of the prior application is considered part of (and is incorporated by reference in) the disclosure of this application.

### BACKGROUND

### 1. Technical Field

This document relates to mounting brackets for medical equipment and methods for their use. For example, this document relates to brackets that can be used to pole mount medical equipment such as blood parameter monitoring devices, extracorporeal blood pumps, and other medical devices.

### 2. Background Information

Clinical settings can often be overcrowded with medical devices and systems that are closely stationed around a patient that is receiving medical treatment. Accordingly, it can be spatially beneficial to mount some medical equipment to a pole or post that is positioned adjacent to or above the patient. The diameters of various medical device mounting poles can be disparate.

Continuous in-line monitoring during cardiopulmonary bypass surgery is a critical component of perfusion safety and improving patient outcomes. Studies have shown that appropriate regulation of blood gas parameters is essential to avoid the negative outcomes linked to sub-optimal blood gas parameter control.

### SUMMARY

This document describes brackets for medical equipment and methods for their use. For example, this document describes brackets that can be used to pole mount medical equipment such as blood parameter monitoring devices, extracorporeal blood pumps, and other medical devices.

In one aspect, a mounting bracket or collar clamp bracket for mounting various medical components and/or devices to medical equipment poles is described herein. The collar clamp bracket can include an ACME helical threaded rod connected to a round hand knob which when turned transfers rotational motion to linear motion of a square nut driven by the ACME threaded rod. The square nut is the clamping/load bearing component which transfers force load to a pivoting clamp jaw.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a perspective view of an example pole clamp mounting bracket assembly that can be used for mounting medical equipment to a pole or post.

FIG. 2 is an exploded view of the pole clamp mounting bracket assembly of FIG. 1.

FIG. 3 is an exploded view of another example of a pole clamp mounting bracket assembly that can be used for mounting medical equipment to a pole or post.

FIG. 4 is a perspective view of an example configuration for using the pole clamp mounting bracket assemblies described herein to pole mount a first type of example medical device.

FIG. 5 is a perspective view of another example configuration for using the pole clamp mounting bracket assemblies described herein to pole mount a second type of example medical device.

FIG. 6 is a perspective view of another example configuration for using the pole clamp mounting bracket assemblies described herein to pole mount a third type of example medical device.

Like reference numbers represent corresponding parts throughout.

### DETAILED DESCRIPTION

This document describes brackets for medical equipment and methods for their use. For example, this document describes brackets that can be used to pole mount medical equipment such as blood parameter monitoring devices, extracorporeal blood pumps, and other medical devices.

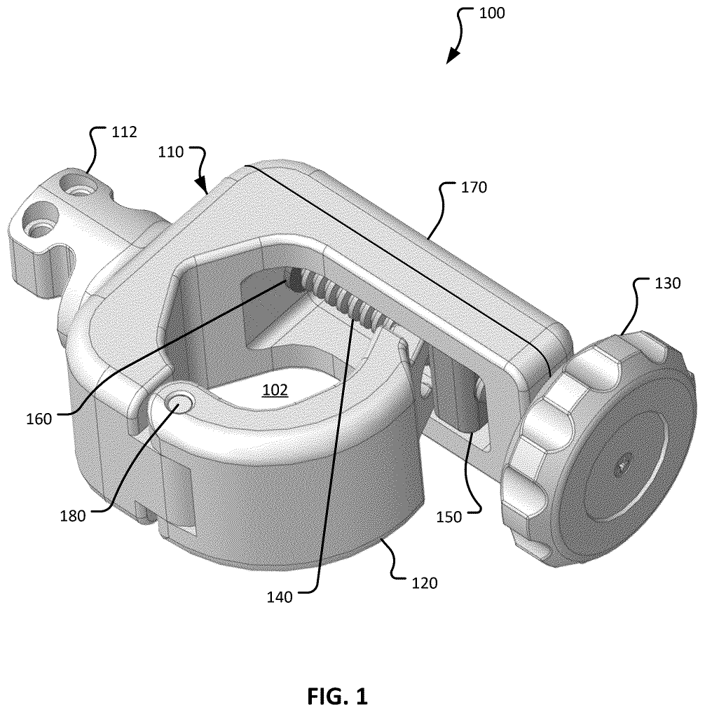

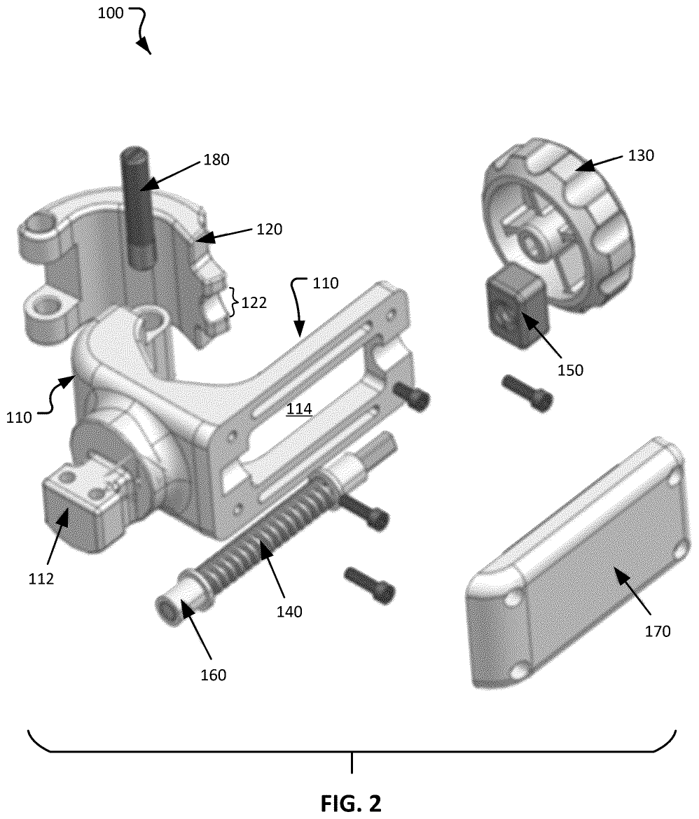

FIGS. 1 and 2 illustrate a first example pole clamp mounting bracket 100 (or simply “bracket 100”). The bracket 100 is configured to be used to pole mount medical equipment such as blood parameter monitoring devices, extracorporeal blood pumps, and other medical devices.

In the depicted embodiment, the bracket 100 includes a main body 110, a clamp jaw 120, an adjustment knob 130, a lead screw 140, an adjustment nut 150, bushings 160, a cover 170, and a pin 180.

The main body 110 is J-shaped. The main body 110 includes an interface coupler 112 that is configurable to be attached to the medical equipment/device to be pole mounted. The interface coupler 112 can be sized and shaped in any desired configuration to interface with, and to be coupled to, any type of medical equipment/device as desired (e.g., patient monitors, pumps, motors, reservoirs, oxygenators, articulating arms, posts, and supports, to provide a few examples).

In the depicted embodiment, the interface coupler 112 has a male “T shape.” The medical device to be attached thereto can have a corresponding female negative shape so that the connection functions like a French cleat or dovetail that initially locks the two together. Also, once these two are attached and easily mounted, the screws can be attached for security.

In some embodiments, the interface coupler 112 is detachable from the main body 110, and another type of interface coupler can be attached to the main body 110.

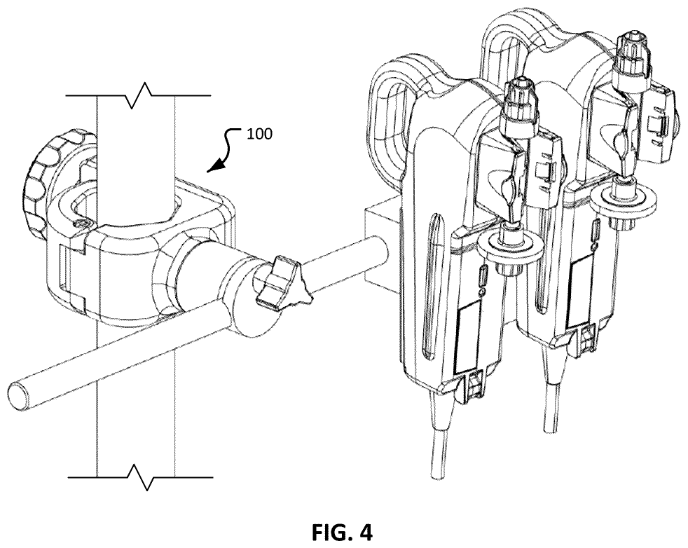

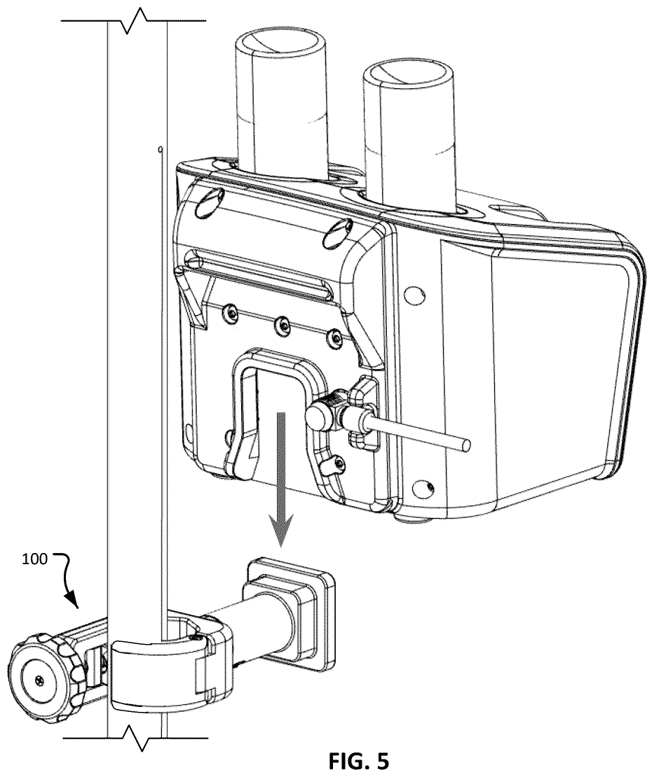

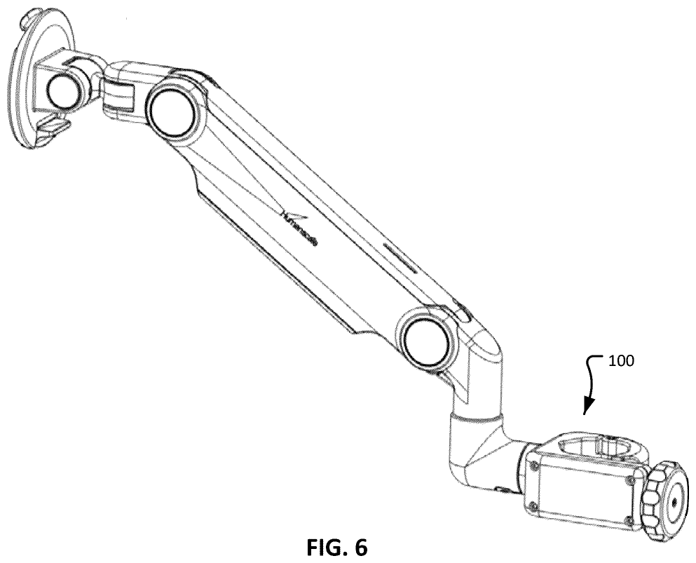

FIGS. 4-6 show a few of examples of how medical devices can be mounted to a pole using the bracket 100. Many other types of medical devices and arrangements or configurations are also possible to be pole mounted using the universal design of the bracket 100.

Still referring to FIGS. 1 and 2, the bracket 100 also includes the clamp jaw 120. The clamp jaw 120 is pivotably coupled to the main body 110 at a revolute joint. The pin 180 is the hinge pin at the revolute joint between the main body 110 and the clamp jaw 120.

The clamp jaw 120 comprises an open slot 122 that slidably receives the lead screw 140. The clamp jaw 120 has an arcuate shape. A peripherally enclosed space 102 (FIG. 1) to receive a pole is defined between the main body 110 and the clamp jaw 120.

The bracket 100 also includes the adjustment knob 130. The size of the space 102 is adjustable (i.e., can be made smaller and larger) by manual rotation of the adjustment knob 130 relative to the main body 110. Accordingly, a clinician user can adjust the bracket 100 to secure it to a pole in order to pole mount a medical device that is attachable to the interface coupler 112 of the bracket 100.

The bracket 100 also includes the lead screw 140. The lead screw 140 is rotatably coupled to the main body 110 and fixedly coupled to the adjustment knob 130. A cover 170 captures the lead screw 140 between the main body 110 and the cover 170. Bushings 160 facilitate low-friction rotations of the lead screw 140 relative to the main body 110.

The bracket 100 also includes the adjustment nut 150. The adjustment nut 150 is threadedly coupled to the lead screw 140 and is translatable along the main body 110 in response to rotation of the adjustment knob 130 relative to the main body 110. A prismatic joint exists between the adjustment nut 150 and the main body 110.

In the depicted embodiment, the adjustment nut 150 is shaped as a rectangular prism. As the lead screw 140 is turned by manual rotation of the adjustment nut 150, the adjustment nut 150 translates/slides along a slot 114 (FIG. 2) defined by the main body 110. The adjustment nut 150 only translates, and does not rotate, because the shape of the slot 114 in relation to the shape of the adjustment nut 150 mechanically prevents the rotation.

As the lead screw 140 is turned by a clinician user who manual rotates the adjustment knob 130, the adjustment nut 150 pushes against the clamp jaw 120 to move the clamp jaw 120 to reduce the space 102. Accordingly, the clamp jaw 120 tightens on a pole that is located within the space 102 by manual rotation of the adjustment nut 150. Reverse rotation of the adjustment nut 150 allows the clamp jaw 120 to move in the opposite direction (to enlarge the space 102). In some embodiments, a spring is included to push the clamp jaw 120 to make it enlarge the space 102. Alternatively, the clinician user can manually move the clamp jaw 120 to enlarge the space 102 when the adjustment nut 150 is backed off from being in contact with the clamp jaw 120.

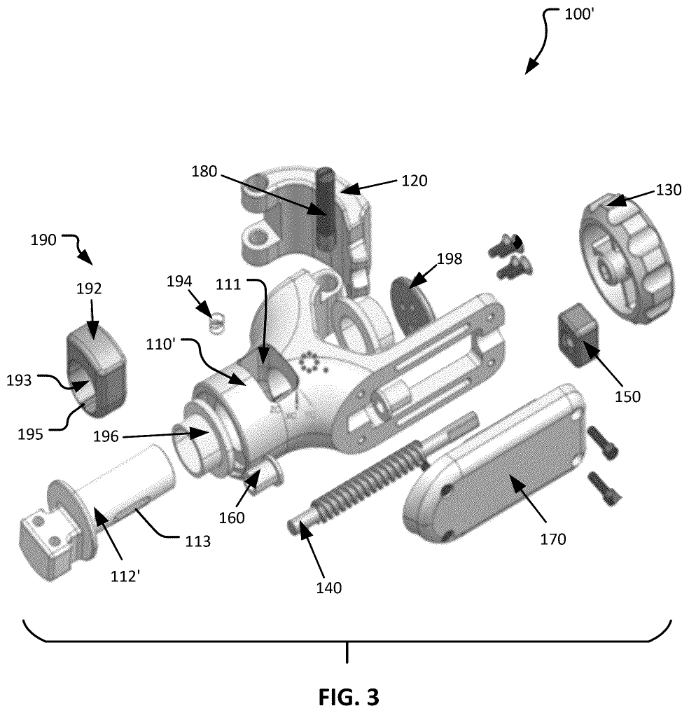

FIG. 3 shows another example pole clamp mounting bracket 100′ (or simply “bracket 100”). The bracket 100′ is configured to be used to pole mount medical equipment such as blood parameter monitoring devices, extracorporeal blood pumps, and other medical devices.

The bracket 100′ is constructed much the same as the bracket 100 (described above in reference to FIGS. 1 and 2) with the exception of the addition of a latch mechanism 190 that allows the interface coupler 112′ to be rotatably coupled to the main body 110′. With the interface coupler 112′ being rotatably coupled to the main body 110′, the medical device attached to the interface coupler 112′ can be advantageously rotated relative to the bracket 100′ (and relative to a pole to which the bracket 100′ is mounted) in multiple different orientations in accordance with a clinician's preferences.

In the depicted embodiment, the latch mechanism 190 includes a depressible spring-loaded button 192 (“button 192”) and a spring 194. The button 192 is movably coupled to the main body 110′ in an opening 111. A spring 194 is positioned between the button 192 and the main body 110′ so that button 192 is biased to a home/latched position (which is upward in FIG. 3).

The button 192 defines an opening 193 through which the interface coupler 112′ extends. On the wall of the opening 193 is a protrusion 195 that extends linearly along the wall of the opening 193. The protrusion 195 can releasably engage in one of the multiple slots 113 defined by the interface coupler 112′. When the button 192 is in its home/latched position, the protrusion 195 is engaged in one of the multiple slots 113 defined by the interface coupler 112′. In that arrangement, the interface coupler 112′ is latched in a particular orientation relative to the main body 110′.

When a clinician user manually depresses the button 192 (and keeps it depressed), the interface coupler 112′ becomes unlatched from the main body 110′ and the interface coupler 112′ can be manually rotated by the clinician user to another orientation relative to the main body 110′. In this context, “unlatched” means that the protrusion 195 is not located in one of the multiple slots 113 defined by the interface coupler 112′. The spring 194 can cause the button 192 to snap into its home/locked position when the protrusion 195 aligns with one of the multiple slots 113 that are defined around the circumference of the interface coupler 112′.

The bracket 100′ also includes bushings 196 that facilitate smooth rotation of the interface coupler 112′ relative to the main body 110′. The bracket 100′ also includes a cover 198 that encloses the interface coupler 112′ relative to the main body 110′.

### Additional Optional Features and Embodiments

In general, the pole clamp mounting bracket described above are capable of mounting various medical components and/or devices to medical equipment poles in a secure and user-friendly manner. In some embodiments, the space 102 can be adjusted to tightly clamp onto poles with outer diameters from 1 inch to 1⅝ inches, or 1 inch to 2 inches, without limitation.

The unique clamping mechanisms of the pole clamp mounting brackets described herein can be comprised of an ACME helical threaded rod (the lead screw 140) connected to a round hand knob (the adjustment knob 130) which, when turned, transfers rotational motion to linear motion of a square nut 150 driven by the ACME threaded rod. The square nut 150 provides the clamping/load bearing component which transfers force load to a pivoting clamp jaw 120.

The benefit of these designs include that fewer rotational turns of the knob are required to impart linear motion of the square nut as compared to current clamping mechanisms. Moreover, the ACME thread gives excellent prevention of back driving of the square nut.

The pole clamp mounting brackets described herein provide a cantilevered arm that supports the attached medical equipment when positioned on a pole. The mechanical joints of the pole clamp mounting bracket described herein are designed to have an appropriate level of friction to dampen free motion to prevent collision of parts. The friction is designed to have ergonomic force thresholds.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

What is claimed is:

1. A bracket assembly for mounting a medical device to a pole, the bracket assembly comprising:
a main body;
a clamp jaw pivotably coupled to the main body, wherein a space to receive the pole is defined between the main body and the clamp jaw;
an adjustment knob;
a lead screw that is rotatably coupled to the main body and fixedly coupled to the adjustment knob; and
an adjustment nut threadedly coupled to the lead screw and translatable along the main body,
wherein the bracket assembly is configured such that rotation of the adjustment knob causes the adjustment nut to cause the clamp jaw to pivot to change the space.

2. The bracket assembly of claim 1, wherein the main body comprises an interface coupler configured to attach to the medical device.

3. The bracket assembly of claim 1, wherein the main body is J-shaped.

4. The bracket assembly of claim 3, wherein the clamp jaw is arcuate.

5. The bracket assembly of claim 1, wherein the clamp jaw comprises an open slot that slidably receives the lead screw.

6. The bracket assembly of claim 1, wherein the adjustment nut is shaped to prevent rotation relative to the main body.

7. The bracket assembly of claim 1, wherein a first end of the clamp jaw is pivotably coupled to the main body, and wherein a second end of the clamp jaw comprises an open slot that slidably receives the lead screw.

8. The bracket assembly of claim 7, wherein the adjustment nut pushes against the second end of the clamp jaw as the adjustment knob is manually turned.

9. A bracket assembly for mounting a medical device to a pole, the bracket assembly comprising:
a main body;
an interface coupler rotatably coupled to the main body and configured to attach to the medical device.
a clamp jaw pivotably coupled to the main body, wherein a space to receive the pole is defined between the main body and the clamp jaw;
an adjustment knob;
a lead screw that is rotatably coupled to the main body and fixedly coupled to the adjustment knob; and
an adjustment nut threadedly coupled to the lead screw and translatable along the main body,
wherein the bracket assembly is configured such that rotation of the adjustment knob causes the adjustment nut to cause the clamp jaw to pivot to change the space.

10. The bracket assembly of claim 9, further comprising a latch mechanism that latches the interface coupler in multiple particular rotational positions relative to the main body.

11. The bracket assembly of claim 10, wherein the latch mechanism comprises a depressible spring-loaded button.

12. The bracket assembly of claim 9, further comprising a cover attached to the main body, and wherein the lead screw is captured between the cover and the main body.

13. The bracket assembly of claim 9, wherein the main body is J-shaped.

14. The bracket assembly of claim 13, wherein the clamp jaw is arcuate.

15. The bracket assembly of claim 9, wherein the clamp jaw comprises an open slot that slidably receives the lead screw.

16. The bracket assembly of claim 9, wherein the adjustment nut is shaped to prevent rotation relative to the main body.

17. The bracket assembly of claim 9, wherein a first end of the clamp jaw is pivotably coupled to the main body, and wherein a second end of the clamp jaw comprises an open slot that slidably receives the lead screw.

18. The bracket assembly of claim 17, wherein the adjustment nut pushes against the second end of the clamp jaw as the adjustment knob is manually turned.
